(19) United States
(12) Patent Application Publication
(10) Pub. No.: US 2009/0005403 A1
(43) Pub. Date: Jan. 1, 2009

(54) Alkinyl-Oxypyrimidines Used in the Form of Pesticides

(75) Inventors: Thomas Bretschneider, Lohmar (DE); Olga Malsam, Rosrath (DE); Mark Wilhelm Drewes, Langenfeld (DE); Christian Arnold, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer Cropscience Aktiengesellschaft, Monheim (DE)

(21) Appl. No.: 11/813,266

(22) PCT Filed: Dec. 24, 2005
(86) PCT No.: PCT/EP2005/014033

§ 371 Date: May 19, 2008

(30) Foreign Application Priority Data
Jan. 5, 2005 (DE) 10 2005 000 747.3

(51) Int. Cl. A01N 43/54; C07D 409/06; C07D 403/06; A01P 7/04; C07D 401/06

(52) U.S. Cl. 514/269; 544/319; 544/296

(57) ABSTRACT

The invention relates to compounds of the formula (I),

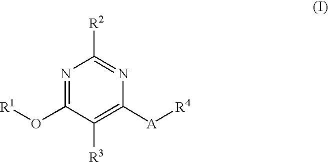

in which
A, R¹, R², R³ and R⁴ are as defined in the description, to processes and intermediates for their preparation and to their use for controlling pests.

The present invention relates to novel substituted pyrimidines, to processes for their preparation and to their use in compositions for controlling animal pests, especially arthropods, in particular insects.

Certain substituted pyrimidines are already known as potential insecticides (cf. WO-2002/024663, WO-2003/076415, WO-2004/085407, WO-2004/099160); however, owing to their action, which is not always satisfactory, they have hitherto not attained any importance.

Certain 4-pyridylpyrimidines are already known as potential fungicides (cf. DE-4031798). However, hitherto nothing has been disclosed with respect to an activity of these compounds against animal pests.

This invention now provides novel substituted pyrimidines of the formula (I)

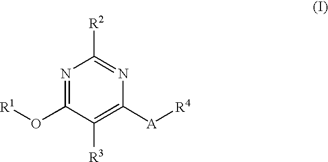

in which
A represents a single bond or represents O (oxygen), S (sulfur), NH, N(C₁-C₄-alkyl), a carbonyl grouping (C═O) or an oxyimino grouping (C═N—O—R, where R represents hydrogen or C₁-C₄-alkyl) or represents optionally hydroxyl- or halogen-substituted straight-chain or branched alkanediyl having 1 to 4 carbon atoms,
R¹ represents optionally halogen-substituted straight-chain or branched alkynyl having 2 to 10 carbon atoms,
R² represents hydrogen, amino, halogen or halogen-substituted straight-chain or branched alkyl having 1 to 4 carbon atoms,
R³ represents hydrogen, amino, halogen or optionally halogen-substituted straight-chain or branched alkyl having 1 to 4 carbon atoms, and
R⁴ represents a monocyclic or bicycyclic heteroaromatic grouping having up to 9 carbon atoms and at least one heteroatom from the group consisting of N (nitrogen, 1 to 5 N atoms), O (oxygen, at most 1 O atom), S (sulfur, at most 1 S atom), which grouping is optionally substituted by nitro, hydroxyl, mercapto, amino, cyano, carboxyl, carbamoyl, thiocarbamoyl, halogen, by in each case optionally hydroxyl-, cyano- or halogen-substituted C₁-C₆-alkyl, C₁-C₆-alkyl-carbonyl, C₁-C₆-alkoxy, C₁-C₆-alkoxy-carbonyl, C₁-C₆-alkylthio, C₁-C₆-alkylsulfinyl, C₁-C₆-alkylsulfonyl, C₁-C₆-alkylamino, C₁-C₆-alkyl-aminocarbonyl, di-(C₁-C₆-alkyl)-amino, di-(C₁-C₆-alkyl)-amino-carbonyl or di-(C₁-C₆-alkyl)-amino-sulfonyl.

Furthermore, it has been found that the novel substituted pyrimidines of the formula (I) are obtained when

(a) reactive pyrimidines of the formula (II)

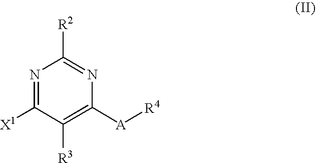

in which
A, R², R³ and R⁴ are as defined above and
X¹ represents halogen or C₁-C₄-alkylsulfonyl,
are reacted with alkynols of the formula (III)

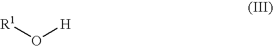

in which
R¹ is as defined above,
   or with alkali metal salts thereof,

if appropriate in the presence of one or more reaction auxiliaries and if appropriate in the presence of one or more diluents,

or when

(b) reactive pyrimidines of the formula (IV)

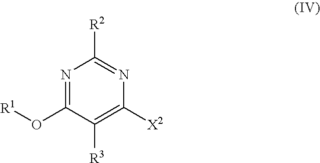

in which
R¹, R² and R³ are as defined above and
X² represents halogen or C₁-C₄-alkylsulfonyl,
are reacted with nucleophilic compounds of the formula (V)

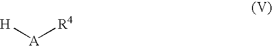

in which
A and R⁴ are as defined above
   or with alkali metal salts thereof,

if appropriate in the presence of one or more reaction auxiliaries and if appropriate in the presence of one or more diluents.

Finally, it has been found that the novel compounds of the formula (I) have strongly pronounced biological properties and are suitable especially for controlling animal pests, in particular insects, arachnids and nematodes, encountered in agriculture, in forests, in the protection of stored products and in the protection of materials and also in the field of hygiene.

The formula (I) provides a general definition of the compounds according to the invention.

Preferred substituents or ranges of the radicals present in the formulae given above and below are illustrated below.
A Preferably represents a single bond or represents O (oxygen), S (sulfur), NH, N(methyl), N(ethyl), N(propyl), a carbonyl grouping (C═O) or an oxyimino grouping (C═N—O—R, where R represents hydrogen, methyl or ethyl) or represents optionally hydroxyl-, fluorine- or chlorine-substituted straight-chain or branched alkanediyl having 1 to 3 carbon atoms.
R¹ Preferably represents optionally fluorine-, chlorine-, bromine- or iodine-substituted straight-chain or branched alkynyl having 3 to 6 carbon atoms.
R² Preferably represents hydrogen, amino, fluorine, chlorine, bromine, iodine or fluorine- and/or chlorine-substituted straight-chain or branched alkyl having 1 to 3 carbon atoms.
R³ Preferably represents hydrogen, amino, fluorine, chlorine, bromine, iodine or optionally fluorine- and/or chlorine-substituted straight-chain or branched alkyl having 1 to 3 carbon atoms.
R⁴ Preferably represents a monocyclic or bicycyclic heteroaromatic grouping having up to 9 carbon atoms and at least one heteroatom from the group consisting of N (nitrogen, 1 to 5 N atoms), O (oxygen, at most 1 O atom), S (sulfur, at most 1 S atom), which grouping is optionally substituted by nitro, hydroxyl, mercapto, amino, cyano, carboxyl, carbamoyl, thiocarbamoyl, fluorine, chlorine, bromine, iodine, by in each case optionally hydroxyl-, cyano-, fluorine-, chlorine- or bromine-substituted C₁-C₅-alkyl, C₁-C₅-alkyl-carbonyl, C₁-C₅-alkoxy, C₁-C₅-alkoxy-carbonyl, C₁-C₅-alkylthio, C₁-C₅-alkylsulfinyl, C₁-C₅-alkyl-sulfonyl, C₁-C₅-alkylamino, C₁-C₅-alkyl-aminocarbonyl, di-(C₁-C₅-alkyl)-amino, di-(C₁-C₅-alkyl)-amino-carbonyl or di-(C₁-C₅-alkyl)-amino-sulfonyl.
   A Particularly preferably represents a single bond or represents O (oxygen), S (sulfur), NH, N(methyl) or represents methylene, ethane-1,1-diyl (ethylidene) or ethane-1,2-diyl (dimethylene).
   R¹ Particularly preferably represents optionally fluorine-, chlorine-, bromine- or iodine-substituted 2-propyn-1-yl, 2-butyn-1-yl, 3-butyn-2-yl or 2-pentyn-1-yl.
   R² Particularly preferably represents hydrogen, fluorine, chlorine, bromine or fluorine- and/or chlorine-substituted methyl.
   R³ Particularly preferably represents hydrogen, fluorine, chlorine, bromine or optionally fluorine- and/or chlorine-substituted methyl.
   R⁴ Particularly preferably represents a monocyclic heteroaromatic grouping from the group consisting of furyl, thienyl, pyrrolyl, pyrazolyl, imidazolyl, oxazolyl, isoxazolyl, thiazolyl, isothiazolyl, oxadiazolyl, thiadiazolyl, triazolyl, pyridinyl, pyrimidinyl, pyrazinyl, pyridazinyl, triazinyl, which grouping is optionally substituted by nitro, hydroxyl, mercapto, amino, cyano, carboxyl, carbamoyl, thiocarbamoyl, fluorine, chlorine, bromine, iodine, by in each case optionally hydroxyl-, cyano-, fluorine- or chlorine-substituted methyl, ethyl, n- or i-propyl, n-, i-, s- or t-butyl, acetyl, propionyl, n- or i-butyroyl, methoxy, ethoxy, n- or i-propoxy, methoxycarbonyl, ethoxycarbonyl, n- or i-propoxycarbonyl, n-, i-, s- or t-butoxycarbonyl, methylthio, ethylthio, n- or i-propylthio, methylsulfinyl, ethylsulfinyl, methylsulfonyl, ethylsulfonyl, methylamino, ethylamino, n- or i-propylamino, n-, i-, s- or t-butylamino, methylaminocarbonyl, ethylaminocarbonyl, n- or i-propylaminocarbonyl, dimethylamino, diethylamino, dimethylaminocarbonyl, diethylaminocarbonyl, dimethylaminosulfonyl or diethylaminosulfonyl.
   A Very particularly preferably represents a single bond or represents O (oxygen), S (sulfur), NH, N(methyl) or methylene.
   R¹ Very particularly preferably represents 2-propyn-1-yl, 2-butyn-1-yl or 2-pentyn-1-yl.
   R² Very particularly preferably represents hydrogen.
   R³ Very particularly preferably represents hydrogen, fluorine, chlorine or methyl.
   R⁴ Very particularly preferably represents a monocyclic heteroaromatic grouping from the group consisting of furyl, thienyl, pyrrolyl, pyrazolyl, imidazolyl, oxazolyl, isoxazolyl, thiazolyl, isothiazolyl, oxadiazolyl, thiadiazolyl, triazolyl, pyridinyl, pyrimidinyl, pyrazinyl, pyridazinyl, triazinyl, which grouping is optionally substituted by nitro, cyano, carboxyl, carbamoyl, thiocarbamoyl, fluorine, chlorine, bromine, by in each case optionally cyano-, fluorine- or chlorine-substituted methyl, ethyl, n- or i-propyl, n-, i-, s- or t-butyl, acetyl, propionyl, n- or i-butyroyl, methoxy, ethoxy, n- or i-propoxy, methoxycarbonyl, ethoxycarbonyl, n- or i-propoxycarbonyl, methylthio, ethylthio, n- or i-propylthio, methylsulfinyl, ethylsulfinyl, methylsulfonyl, ethylsulfonyl, methylamino, ethylamino, n- or i-propylamino, methylaminocarbonyl, ethylaminocarbonyl, n- or i-propylaminocarbonyl, dimethylamino, diethylamino, dimethylaminocarbonyl or dimethylaminosulfonyl.

Preference according to the invention is given to the compounds of the formula (I) which contain the combinations of the meanings listed above as being preferred.

Particular preference according to the invention is given to the compounds of the formula (I) which contain the combinations of the meanings listed above as being particularly preferred.

Very particular preference according to the invention is given to the compounds of the formula (I) which contain the combinations of the meanings listed above as being very particularly preferred.

In the radical definitions given above and below, hydrocarbon radicals, such as alkyl or alkynyl, are in each case straight-chain or branched as far as this is possible—including in combination with heteroatoms, such as alkoxy or alkynyloxy.

Using, for example, 4-chloro-5-fluoro-6-(pyrazol-1-yl)pyrimidine and 2-propyn-1-ol as starting materials, the course of the reaction in the preparation process (a) according to the invention can be illustrated by the formula scheme below:

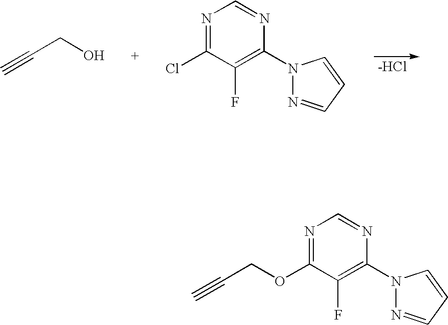

Using, for example, 4-fluoro-6-(2-butyn-1-yl-oxy)pyrimidine and imidazole as starting materials, the course of the reaction in the preparation process (b) according to the invention can be illustrated by the formula scheme below:

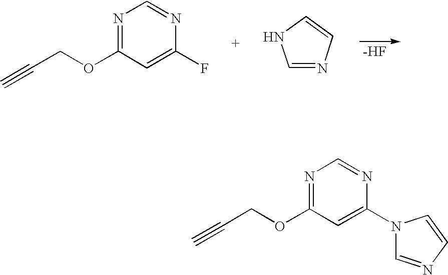

The formula (II) provides a general definition of the reactive pyrimidines to be used as starting materials in the process (a) according to the invention for preparing the compounds of the general formula (I). In the formula (II), A, R², R³ and R⁴ preferably or in particular have those meanings which have already been mentioned above, in connection with the description of the compounds of the general formula (I) according to the invention, as being preferred or as being particularly preferred for A, R², R³ and R⁴; X¹ preferably represents fluorine, chlorine, bromine, methylsulfonyl or ethylsulfonyl, in particular chlorine.

The starting materials of the general formula (II) are known and/or can be prepared by processes known per se (cf. DE-4031798).

The formula (III) provides a general definition of the alkynols further to be used as starting materials in the process (a) according to the invention for preparing the compounds of the general formula (I). In the formula (III), R¹ preferably or in particular has that meaning which has already been mentioned above, in connection with the description of the compounds of the general formula (I) according to the invention, as being preferred or as being particularly preferred for R¹.

The starting materials of the formula (III) are known organic chemicals for synthesis.

The formula (IV) provides a general definition of the reactive pyrimidines to be used as starting materials in the process (b) according to the invention for preparing the compounds of the general formula (I). In the formula (IV), R¹, R² and R³ preferably or in particular have those meanings which have already been mentioned above, in connection with the description of the compounds of the general formula (I) according to the invention, as being preferred or as being particularly preferred for R¹, R² and R³; X² preferably represents fluorine, chlorine, bromine, methylsulfonyl or ethylsulfonyl, in particular chlorine.

The starting materials of the formula (IV) are known and/or can be prepared by processes known per se (cf. WO-2002/024663).

The formula (V) provides a general definition of the nucleophilic compounds further to be used as starting materials in the process (b) according to the invention for preparing the compounds of the general formula (I). In the formula (V), A and R⁴ preferably or in particular have those meanings which have already been mentioned above, in connection with the description of the compounds of the general formula (I) according to the invention, as being preferred or as being particularly preferred for A and R⁴.

The processes (a) and (b) according to the invention for preparing the compounds of the formula (I) are preferably carried out using a plurality of reaction auxiliaries. Suitable for use as reaction auxiliaries for carrying out the processes (a) and (b) according to the invention are all suitable inorganic or organic acid acceptors. These preferably include alkali metal and alkaline earth metal compounds and also basic nitrogen compounds, in particular alkylamines. Examples which may be mentioned are the hydrides, hydroxides, oxides and carbonates of lithium, sodium, potassium, magnesium, calcium, barium and cesium, furthermore further basic compounds, such as amidine bases or guanidine bases, such as 7-methyl-1,5,7-triazabicyclo(4.4.0)dec-5-ene (MTBD), diazabicyclo(4.3.0)nonene (DBN), diazabicyclo(2.2.2)octane (DABCO), 1,8-diazabicyclo-(5.4.0)undecene (DBU), cyclohexyltetrabutylguanidine (CyTBG), cyclohexyltetramethylguanidine (CyTMG), N,N,N,N-tetramethyl-1,8-naphthalenediamine, pentamethylpiperidine, tertiary amines, such as triethylamine, trimethylamine, N-ethyldiisopropylamine, tribenzylamine, triisobutylamine, tributylamine, tribenzylamine, N,N-dimethylcyclohexylamine, N-ethyldicyclohexylamine, triamylamine, trihexylamine, N,N-dimethylaniline, N,N-dimethyltoluidine, N,N-dimethyl-p-aminopyridine, N-methylpyrrolidine, N-methylpiperidine, N-methylimidazole, N-methylpyrrole, N-methylmorpholine, N-methylhexamethyleneimine, pyridine, 4-pyrrolidinopyridine, 4-dimethyl-aminopyridine, quinoline, α-picoline, β-picoline, isoquinoline, pyrimidine, acridine, N,N,N′,N′-tetramethylenediamine, N,N,N′,N′-tetraethylenediamine, quinoxaline, N-propyldiisopropylamine, N-ethyldiisopropylamine, N,N′-dimethylcyclohexylamine, 2,6-lutidine, 2,4-lutidine or triethylenediamine.

Also suitable for use as reaction auxiliaries are, if appropriate, Lewis acids, such as, for example, aluminum chloride, boric acid, boron trifluoride, trimethylstannane, zinc(II) chloride, zinc(II) bromide or compounds having similar properties.

The processes (a) and (b) according to the invention for preparing the compounds of the formula (I) are preferably carried out using or a plurality of diluents. Suitable diluents for carrying out the processes (a) and (b) according to the invention are all solvents which are inert under the reaction conditions. Examples which may be mentioned are: halogenated hydrocarbons, in particular chlorinated hydrocarbons, such as tetrachloroethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene; alcohols, such as methanol, ethanol, isopropanol, butanol; ethers, such as ethyl propyl ether, methyl tert-butyl ether, n-butyl ether, anisole, phenethol, cyclohexyl methyl ether, dimethyl ether, diethyl ether, dipropyl ether, diisopropyl ether, di-n-propyl ether, diisobutyl ether, diisoamyl ether, ethylene glycol dimethyl ether, tetrahydrofuran, dioxane, dichlorodiethyl ether and polyethers of ethylene oxide and/or propylene oxide; amines, such as trimethylamine, triethylamine, tripropylamine, tributylamine, N-methylmorpholine, pyridine and tetramethylenediamine, nitrated hydrocarbons, such as nitromethane, nitroethane, nitropropane, nitrobenzene, chloronitrobenzene, o-nitrotoluene; nitriles, such as acetonitrile, propionitrile, butyronitrile, isobutyronitrile, benzonitrile, m-chloro-benzonitrile, and also compounds such as tetrahydrothiophene oxide and dimethyl sulfoxide, tetramethyl sulfoxide, dipropyl sulfoxide, benzyl methyl sulfoxide, diisobutyl sulfoxide, dibutyl sulfoxide, diisoamyl sulfoxide; sulfones, such as dimethyl sulfone, diethyl sulfone, dipropyl sulfone, dibutyl sulfone, diphenyl sulfone, dihexyl sulfone, methyl hexyl sulfone, ethyl propyl sulfone, ethyl isobutyl sulfone and pentamethylene sulfone; aliphatic, cycloaliphatic or aromatic hydrocarbons, for example white spirits with components having boiling points in the range of, for example, from 40° C. to 250° C., cymene, petroleum fractions within a boiling point interval of from 70° C. to 190° C., cyclohexane, methylcyclohexane, petroleum ether, ligroin, octane, benzene, toluene, chlorobenzene, bromobenzene, nitrotoluene, xylene; esters, such as methyl acetate, ethyl acetate, butyl acetate, isobutyl acetate, and also dimethyl carbonate, dibutyl carbonate, ethylene carbonate; amides, such as hexamethylenephosphoric triamide, formamide, N-methylformamide, N,N-dimethylformamide, N,N-dipropylformamide, N,N-dibutylformamide, N-methylpyrrolidine, N-methylcaprolactam, 1,3-dimethyl-3,4,5,6-tetrahydro-2(1H)pyrimidine, octylpyrrolidine, octyl-caprolactam, 1,3-dimethyl-2-imidazolinedione, N-formylpiperidine, N,N′-1,4-diformylpiperazine; ketones, such as acetone, acetophenone, methyl ethyl ketone, methyl butyl ketone, water.

When carrying out the process according to the invention, the reaction temperatures can be varied within a relatively wide range. In general, the process is carried out at temperatures between −50° C. and +150° C., preferably between −20° C. and +120° C.

The process according to the invention is generally carried out under atmospheric pressure. However, it is also possible to carry out the process according to the invention under elevated or reduced pressure—in general between 0.1 bar and 15 bar.

For carrying out the process according to the invention, the starting materials are generally employed in approximately equimolar amounts. However, it is also possible to use a relatively large excess of one of the components. The reaction is generally carried out in a suitable diluent in the presence of a reaction auxiliary, if appropriate also under an atmosphere of protective gas (for example under nitrogen, argon or helium), and the reaction mixture is generally stirred at the required temperature for a number of hours. Work-up is carried out by customary methods (cf. the Preparation Examples).

The active compounds of the formula (I) according to the invention are well tolerated by plants, have favorable homeotherm toxicity and are environmentally friendly; they are suitable for protecting plants and plant organs, for increasing harvest yields, for improving the quality of the harvested material and for controlling animal pests, in particular insects, arachnids and nematodes encountered in agriculture, in forests, in gardens and leisure facilities, in the protection of stored products and in the protection of materials and in the field of hygiene. They are preferably used as crop protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae and Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp., Damalinia spp.

From the order of the Thysanoptera, for example, Hercinothrips femoralis, Thrips tabaci, Thrips palmi, Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium comi, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp. and Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomrma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The plant-parasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp. and Bursaphelenchus spp.

The compounds of the formula (I) according to the invention are distinguished in particular by strong activity against aphids, for example Aphis spp. and Myzus spp.

If appropriate, the compounds according to the invention may also be used in certain concentrations or application rates to act as herbicides or as safeners for this purpose, or as microbicides, for example as fungicides, antimycotics and bactericides. If appropriate, they can also be employed as intermediates or precursors for the synthesis of further active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

The treatment according to the invention of the plants and plant parts with the active compounds is carried out directly or by allowing the compounds to act on their surroundings, habitat or storage space by the customary treatment methods, for example by immersion, spraying, evaporation, fogging, scattering, painting on, injecting and, in the case of propagation material, in particular in the case of seeds, also by applying one or more coats.

The active compounds can be converted into the customary formulations such as solutions, emulsions, wettable powders, suspensions, powders, dusts, pastes, soluble powders, granules, suspension-emulsion concentrates, natural and synthetic materials impregnated with active compound, and microencapsulations in polymeric materials.

These formulations are produced in a known manner, for example by mixing the active compounds with extenders, that is, liquid solvents and/or solid carriers, optionally with the use of surfactants, that is, emulsifiers and/or dispersants, and/or foam formers.

If the extender used is water, it is also possible, for example, to use organic solvents as cosolvents. The following are essentially suitable as liquid solvents: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, or else water.

Suitable solid carriers are:

for example ammonium salts and ground natural minerals such as kaolins, clays, talc, chalk, quartz, attapulgite, montmorillonite or diatomaceous earth, and ground synthetic minerals such as highly disperse silica, alumina and silicates; suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks; suitable emulsifiers and/or foam formers are: for example nonionic and anionic emulsifiers such as polyoxyethylene fatty acid esters, polyoxyethylene fatty alcohol ethers, for example alkylaryl polyglycol ethers, alkylsulfonates, alkyl sulfates, arylsulfonates, or else protein hydrolysates; suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

The active compound according to the invention can be present in its commercially available formulations and in the use forms prepared from these formulations as a mixture with other active compounds, such as insecticides, attractants, sterilants, bactericides, acaricides, nematicides, fungicides, growth regulators or herbicides. The insecticides include, for example, phosphoric esters, carbamates, carboxylic esters, chlorinated hydrocarbons, phenylureas, substances produced by microorganisms, etc.

Particularly favorable mixing partners are, for example, the following:

Fungicides:

2-phenylphenol; 8-hydroxyquinoline sulfate; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methylsulfonyl)amino]butanamide; 1-(1-naphthalenyl)-1H-pyrrole-2,5-dione; 2,3,5,6-tetra-chloro-4-(methylsulfonyl)pyridine; 2-amino4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridine-dicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxaspiro[4.5]decane-3-amine; sodium tetrathiocarbonate;

and copper salts and preparations, such as Bordeaux mixture; copper hydroxide; copper naphthenate; copper oxychloride; copper sulfate; cufraneb; cuprous oxide; mancopper; oxine-copper.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

### Insecticides/Acaricides/Nematicides:

1. Acetylcholine esterase (AChE) inhibitors

1.1 Carbamates, for example
alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb triazamates

1.2 Organophosphates, for example
acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulfone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion

2. Sodium channel modulators / voltage-dependent sodium channel blockers

2.1 Pyrethroids, for example
acrinathrin, allethrin (d-cis-trans, d-trans), beta-cyfluthrin, bifenthrin, bioallethrin, bioallethrin-S cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum)

DDT

2.2 Oxadiazines, for example indoxacarb

3. Acetylcholine receptor agonists/antagonists

3.1 Chloronicotinyls, for example
acetamiprid, clothianidin, dinotefuran, imidacloprid, nitenpyram, nithiazine, thiacloprid, thiamethoxam

3.2 Nicotine, bensultap, cartap

4. Acetylcholine receptor modulators

4.1. Spinosyns, for example spinosad

5. GABA-controlled chloride channel antagonists

5.1 Cyclodiene organochlorines, for example
camphechlor, chlordane, endosulfan, gamma-HCH, HCH, heptachlor, lindane, methoxychlor

5.2 Fiproles, for example
acetoprole, ethiprole, fipronil, vaniliprole

6. Chloride channel activators

6.1 Mectins, for example
avermectin, emamectin, emamectin-benzoate, ivermnectin, milbemycin

7. Juvenile hormone mimetics, for example
diofenolan, epofenonane, fenoxycarb, hydroprene, kinoprene, methoprene, pyriproxifen, triprene

8. Ecdyson agonists/disruptors

8.1 Diacylhydrazines, for example
chromafenozide, halofenozide, methoxyfenozide, tebufenozide

9. Chitin biosynthesis inhibitors

9.1 Benzoylureas, for example
bistrifluron, chlofluazuron, diflubenzuron, fluazuron, flucycloxuron, flufenoxuron, hexaflumuron, lufenuron, novaluron, noviflumuron, penfluron, teflubenzuron, triflumuron

9.2 Buprofezin

9.3 Cyromazine

10. Oxidative phosphorylation inhibitors, ATP disruptors

10.1 Diafenthiuron

10.2 Organotins, for example azocyclotin, cyhexatin, fenbutatin-oxide

11. Oxidative phosphorylation decouplers acting by interrupting the H-proton gradient

11.1 Pyrroles, for example chlorfenapyr

11.2 Dinitrophenols, for example binapacyrl, dinobuton, dinocap, DNOC

12. Side-I electron transport inhibitors

12.1 METIs, for example fenazaquin, fenpyroximate, pyrimidifen, pyridaben, tebufenpyrad, tolfenpyrad

12.2 Hydramethylnon

12.3 Dicofol

13. Side-II electron transport inhibitors
Rotenone

14. Side-III electron transport inhibitors
Acequinocyl, fluacrypyrim

15. Microbial disruptors of the insect gut membrane
Bacillus thuringiensis strains

16. Fat synthesis inhibitors

16.1 Tetronic acids, for example
spirodiclofen, spiromesifen

16.2 Tetramic acids, for example
3-(2,5-dimethylphenyl)-8-methoxy-2-oxo-1-azaspiro[4.5]dec-3-en4-yl ethyl carbonate (aka: carbonic acid, 3-(2,5-dimethylphenyl)-8-methoxy-2-oxo-1-azaspiro[4.5]dec-3-en-4-yl ethyl ester, CAS-Reg.-No.: 382608-10-8) and carbonic acid, cis-3-(2,5-dimethylphenyl)-8-methoxy-2-oxo-1-azaspiro[4.5]dec-3-en-4-yl ethyl ester (CAS-Reg.-No.: 203313-25-1)

17. Carboxamides, for example flonicamid

18. Octopaminergic agonists, for example amitraz

19. Inhibitors of magnesium-stimulated ATPase, for example propargite

20. BDCAs, for example N2-[1,1-dimethyl-2-(methylsulfonyl)ethyl]-3-iodo-N1-[2-methyl-4-[1,2,2,2-tetrafluoro-1-(trifluoromethyl)ethyl]phenyl]-1,2-benzenedicarboxamide (CAS-Reg.-No.: 272451-65-7)

21. Nereistoxin analogs, for example thiocyclam hydrogen oxalate, thiosultap-sodium

22. Biologicals, hormones or pheromones, for example
azadirachtin, Bacillus spec., Beauveria spec., codlemone, Metarrhizium spec., Paecilomyces spec., thuringiensin, Verticillium spec.

23. Active compounds with unknown or unspecific mechanisms of action

23.1 Fumigants, for example
aluminum phosphide, methyl bromide, sulfuryl fluoride

23.2 Selective antifeedants, for example
cryolite, flonicamid, pymetrozine

23.3 Mite growth inhibitors, for example
clofentezine, etoxazole, hexythiazox

23.4 Amidoflumet, benclothiaz, benzoximate, bifenazate, bromopropylate, buprofezin, quino-methionate, chlordimeform, chlorobenzilate, chloropicrin, clothiazoben, cycloprene, cyflumetofen, dicyclanil, fenoxacrim, fentrifanil, flubendiamide, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin

and also products which comprise insecticidal plant extracts, nematodes, fungi or viruses.

A mixture with other known active compounds, such as herbicides, or with fertilizers and growth regulators, safeners and/or semiochemicals is also possible.

When used as insecticides in their commercially available formulations and in the use forms prepared with these formulations, the active compounds according to the invention can furthermore be present in the form of a mixture with synergists. Synergists are compounds by which the activity of the active compounds is increased without it being necessary for the synergist added to be active itself.

When used as insecticides in their commercially available formulations and in the use forms prepared with these formulations, the active compounds according to the invention can furthermore be present in the form of a mixture with inhibitors which reduce the degradation of the active compound after application in the habitat of the plant, on the surface of parts of plants or in plant tissues.

The active compound content of the use forms prepared from the commercially available formulations can vary within broad ranges. The active compound concentration of the use forms can be from 0.0000001 up to 95% by weight of active compound, preferably between 0.0001 and 1% by weight.

They are applied in a customary manner adapted to suit the use forms.

When used against hygiene pests and pests of stored products, the active compound is distinguished by excellent residual action on wood and clay as well as good stability to alkali on limed substrates.

As already mentioned above, it is possible to treat all plants and their parts in accordance with the invention. In a preferred embodiment, wild plant species or plant varieties and plant cultivars which have been obtained by conventional biological breeding methods, such as hybridization or protoplast fusion, and the parts of these varieties and cultivars are treated. In a further preferred embodiment, transgenic plants and plant cultivars which have been obtained by recombinant methods, if appropriate in combination with conventional methods (genetic modified organisms), and their parts are treated. The term “parts” or “parts of plants” or “plant parts” has been explained above.

Plants which are treated particularly preferably in accordance with the invention are those of the plant cultivars which are in each case commercially available or in use. Plant cultivars are understood as meaning plants with new traits which have been bred either by conventional breeding, by mutagenesis or by recombinant DNA techniques. They may take the form of cultivars, biotypes and genotypes.

Depending on the plant species or plant cultivars, their location and growth conditions (soils, climate, vegetation period, nutrition), the treatment according to the invention may also result in superadditive (“synergistic”) effects. Thus, for example, reduced application rates and/or a widened activity spectrum and/or an increase in the activity of the substances and compositions which can be used in accordance with the invention, better plant growth, increased tolerance to high or low temperatures, increased tolerance to drought or to salinity in the water or soil, increased flowering performance, facilitated harvesting, accelerated maturation, higher yields, higher quality and/or better nutritional value of the harvested products, better storage characteristics and/or processability of the harvested products are possible which exceed the effects which were actually to be expected.

The preferred transgenic plants or plant cultivars (those obtained by recombinant methods) to be treated in accordance with the invention include all those plants which, owing to the process of recombinant modification, were given genetic material which confers particular, advantageous, valuable traits to these plants. Examples of such properties are better plant growth, increased tolerance to high or low temperatures, increased tolerance to drought or to salinity in the water or soil, increased flowering performance, facilitated harvesting, accelerated maturation, higher yields, higher quality and/or higher nutritional value of the harvested products, better storage characteristics and/or processability of the harvested products. Further examples of such traits, examples which must be mentioned especially, are better defense of the plants against animal and microbial pests, such as against insects, mites, phytopathogenic fungi, bacteria and/or viruses and an increased tolerance of the plants to certain herbicidal active compounds. Examples of transgenic plants which may be mentioned are the important crop plants, such as cereals (wheat, rice), maize, soybeans, potato, cotton, tobacco, oilseed rape and fruit plants (with the fruits apples, pears, citrus fruits and grapes), with particular emphasis on maize, soybeans, potato, cotton, tobacco, and oilseed rape. Traits which are especially emphasized are the increased defense of the plants against insects, arachnids, nematodes and slugs and snails, owing to toxins being formed in the plants, in particular toxins which are generated in the plants by the genetic material of Bacillus thuringiensis (for example by the genes CryIA(a), CryIA(b), CryIA(c), CryIIA, CryIIIA, CryIIIB2, Cry9c Cry2Ab, Cry3Bb and CryIF and their combinations; hereinbelow “Bt plants”). Other traits which are particularly emphasized are the increased defense of plants against fungi, bacteria and viruses by the systemic acquired resistance (SAR), systemin, phytoalexins, elicitors and resistance genes and correspondingly expressed proteins and toxins. Other traits which are especially emphasized are the increased tolerance of the plants to certain herbicidal active compounds, for example imidazolinones, sulfonylureas, glyphosate or phosphinotricin (for example “PAT” gene). The genes which confer the desired traits in each case may also be present in the transgenic plants in combination with one another. Examples of “Bt plants” which may be mentioned are maize cultivars, cotton cultivars, soybean cultivars and potato cultivars which are commercially available under the trade names YIELD GARD® (for example maize, cotton, soybeans), KnockOut® (for example maize), StarLink® (for example maize), Bollgard® (cotton), Nucotn® (cotton) and NewLeaf(® (potato). Examples of herbicide-tolerant plants which may be mentioned are maize cultivars, cotton cultivars and soybean cultivars which are commercially available under the trade names Roundup Ready® (tolerance to glyphosate, for example maize, cotton, soybean), Liberty Link® (tolerance to phosphinotricin, for example oilseed rape), IMI® (tolerance to imidazolinones) and STS® (tolerance to sulfonylureas, for example maize). Herbicide-resistant plants (plants bred in a conventional manner for herbicide tolerance) which may be mentioned also include the varieties commercially available under the name Clearfield® (for example maize). Naturally, these statements also apply to plant cultivars having these genetic traits or genetic traits still to be developed, which plant cultivars will be developed and/or marketed in the future.

The plants listed can be treated particularly advantageously according to the invention with the compounds of the general formula I or the active compound mixtures according to the invention. The preferred ranges stated above for the active compounds and mixtures also apply to the treatment of these plants. Particular emphasis may be given to the treatment of plants with the compounds or mixtures specifically mentioned in the present text.

The active compounds according to the invention are not only active against plant, hygiene and stored-product pests, but also, in the veterinary medicine sector, against animal parasites (ectoparasites), such as ixodid ticks, argasid ticks, scab mites, trombi-culid mites, flies (stinging and licking), parasitic fly larvae, lice, hair lice, bird lice and fleas. These parasites include:

From the order of the Anoplurida, for example, Haematopinus spp., Linognathus spp., Pediculus spp., Phtirus spp., Solenopotes spp.

From the order of the Mallophagida and the sub-orders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp.

From the order of the Diptera and the sub-orders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopyslla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattela germanica and Supella spp.

From the sub-class of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which attack agricultural livestock, such as, for example, cattle, sheep, goats, horses, pigs, donkeys, camels, buffaloes, rabbits, chickens, turkeys, ducks, geese, honeybees, other domestic animals, such as, for example, dogs, cats, cage birds, aquarium fish, and so-called experimental animals, such as, for example, hamsters, guinea-pigs, rats and mice. By combating these arthropods, it is intended to reduce deaths and decreased performances (in meat, milk, wool, hides, eggs, honey and the like), so that more economical and simpler animal keeping is made possible by using the active compounds according to the invention.

In the veterinary sector, the active compounds according to the invention are used in a known manner by enteral administration, for example in the form of tablets, capsules, drinks, drenches, granules, pastes, boli, the feed-through method, suppositories, by parenteral administration, such as, for example, by means of injections (intramuscular, subcutaneous, intravenous, intraperitoneal and the like), implants, by nasal application, by dermal administration, for example in the form of dipping or bathing, spraying, pouring-on and spotting-on, washing, dusting, and with the aid of shaped articles which comprise active compound, such as collars, ear tags, tail marks, limb bands, halters, marking devices and the like.

When administered to livestock, poultry, domestic animals and the like, the active compounds of the formula (I) can be used as formulations (for example powders, emulsions, flowables) which comprise the active compounds in an amount of 1 to 80% by weight, either directly or after dilution by a factor of 100 to 10 000, or they may be used in the form of a chemical bath.

Furthermore, it has been found that the compounds according to the invention have a potent insecticidal action against insects which destroy industrial materials.

The following insects may be mentioned by way of example and as being preferred, but without any limitation:

Beetles, such as Hylotrupes bajulus, Chlorophorus pilosis, Anobium punctatum, Xestobium rufovillosum, Ptilinus pecticomis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutus.

Dermapterans, such as Sirex juvencus, Urocerus gigas, Urocerus gigas taignus, Urocerus augur.

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus.

Bristletails, such as Lepisma saccharina.

Industrial materials are to be understood as meaning, in the present context, non-live materials, such as, preferably, synthetic materials, glues, sizes, paper and board, leather, wood and timber products, and paint.

The materials to be very particularly preferably protected against attack by insects are wood and timber products.

Wood and timber products which can be protected by the composition according to the invention or mixtures comprising such a composition are to be understood as meaning, for example:

construction timber, wooden beams, railway sleepers, bridge components, jetties, wooden vehicles, boxes, pallets, containers, telephone poles, wood cladding, windows and doors made of wood, plywood, particle board, joiner's articles, or wood products which, quite generally, are used in the construction of houses or in joinery.

The active compounds can be used as such, in the form of concentrates or generally customary formulations, such as powders, granules, solutions, suspensions, emulsions or pastes.

The formulations mentioned can be prepared in a manner known per se, for example by mixing the active compounds with at least one solvent or diluent, emulsifier, dispersant and/or binder or fixative, water repellent, if appropriate desiccants and UV stabilizers and, if appropriate, colorants and pigments and other processing auxiliaries.

The insecticidal compositions or concentrates used for the protection of wood and wooden materials comprise the active compound according to the invention in a concentration of 0.0001 to 95% by weight, in particular 0.001 to 60% by weight.

The amount of the compositions or concentrates employed depends on the species and the occurrence of the insects and on the medium. The optimum rate of application can be determined upon use in each case by a test series. However, in general, it suffices to employ 0.0001 to 20% by weight, preferably 0.001 to 10% by weight, of the active compound, based on the material to be protected.

The solvent and/or diluent used is an organochemical solvent or solvent mixture and/or an oily or oil-type organochemical solvent or solvent mixture of low volatility and/or a polar organochemical solvent or solvent mixture and/or water and, if appropriate, an emulsifier and/or wetting agent.

Organochemical solvents which are preferably employed are oily or oil-type solvents having an evaporation number of above 35 and a flashpoint of above 30° C., preferably above 45° C. Substances which are used as such oily and oil-type solvents which have low volatility and are insoluble in water are suitable mineral oils or their aromatic fractions, or mineral-oil-containing solvent mixtures, preferably white spirit, petroleum and/or alkylbenzene.

Substances which are advantageously used are mineral oils with a boiling range of 170 to 220° C., white spirit with a boiling range of 170 to 220° C., spindle oil with a boiling range of 250 to 350° C., petroleum or aromatics of boiling range 160 to 280° C., essence of terpentine and the like.

In a preferred embodiment, liquid aliphatic hydrocarbons with a boiling range of 180 to 210° C. or high-boiling mixtures of aromatic and aliphatic hydrocarbons with a boiling range of 180 to 220° C. and/or spindle oil and/or monochloronaphthalene, preferably α-monochloronaphthalene, are used.

The organic oily or oil-type solvents of low volatility having an evaporation number of above 35 and a flashpoint of above 30° C., preferably above 45° C., can be partially replaced by organochemical solvents of high or medium volatility, with the proviso that the solvent mixture also has an evaporation number of above 35 and a flashpoint of above 30° C., preferably above 45° C., and that the insecticide/fungicide mixture is soluble or emulsifiable in this solvent mixture.

In a preferred embodiment, part of the organochemical solvent or solvent mixture or an aliphatic polar organochemical solvent or solvent mixture is replaced. Substances which are preferably used are aliphatic organochemical solvents having hydroxyl and/or ester and/or ether groups, such as, for example, glycol ethers, esters and the like.

The organochemical binders used within the scope of the present invention are the synthetic resins and/or binding drying oils which are known per se and can be diluted with water and/or are soluble or dispersible or emulsifiable in the organochemical solvents employed, in particular binders composed of, or comprising, an acrylate resin, a vinyl resin, for example polyvinyl acetate, polyester resin, polycondensation or polyaddition resin, polyurethane resin, alkyd resin or modified alkyd resin, phenol resin, hydrocarbon resin, such as indene/coumarone resin, silicone resin, drying vegetable and/or drying oils and/or physically drying binders based on a natural and/or synthetic resin.

The synthetic resin used as the binder can be employed in the form of an emulsion, dispersion or solution. Up to 10% by weight of bitumen or bituminous substances can also be used as binders. In addition, colorants, pigments, water repellents, odor-masking substances and inhibitors or anticorrosives known per se and the like can also be employed.

The composition or the concentrate preferably comprises, in accordance with the invention, at least one alkyd resin or modified alkyd resin and/or a drying vegetable oil as the organochemical binder. Preferably used according to the invention are alkyd resins with an oil content of over 45% by weight, preferably 50 to 68% by weight.

All or some of the abovementioned binder can be replaced by a fixative (mixture) or a plasticizer (mixture). These additives are intended to prevent volatilization of the active compounds and crystallization or precipitation. They preferably replace 0.01 to 30% of the binder (based on 100% of binder employed).

The plasticizers are from the chemical classes of the phthalic esters, such as dibutyl phthalate, dioctyl phthalate or benzyl butyl phthalate, the phosphoric esters, such as tributyl phosphate, the adipic esters, such as di(2-ethylhexyl) adipate, the stearates, such as butyl stearate or amyl stearate, the oleates, such as butyl oleate, the glycerol ethers or relatively high-molecular-weight glycol ethers, glycerol esters and p-toluenesulfonic esters.

Fixatives are chemically based on polyvinyl alkyl ethers, such as, for example, polyvinyl methyl ether, or ketones, such as benzophenone or ethylenebenzophenone.

Particularly suitable as a solvent or diluent is also water, if appropriate as a mixture with one or more of the abovementioned organochemical solvents or diluents, emulsifiers and dispersants.

Particularly effective protection of wood is achieved by large-scale industrial impregnation processes, for example vacuum, double-vacuum or pressure processes.

If appropriate, the ready-to-use compositions can additionally comprise other insecticides and, if appropriate, additionally one or more fungicides.

Suitable additional components which may be admixed are, preferably, the insecticides and fungicides mentioned in WO 94/29 268. The compounds mentioned in that document are expressly part of the present application.

Very particularly preferred components which may be admixed are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyfenozide, triflumuron, chlothianidin, spinosad, tefluthrin,

and fungicides, such as epoxyconazole, hexaconazole, azaconazole, propiconazole, tebuconazole, cyproconazole, metconazole, imazalil, dichlofluanid, tolylfluanid, 3-iodo-2-propynylbutyl carbamate, N-octyl-isothiazolin-3-one and 4,5-dichloro-N-octylisothiazolin-3-one.

The compounds according to the invention can at the same time be employed for protecting objects which come into contact with salt water or brackish water, in particular hulls, screens, nets, buildings, moorings and signaling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis(trialkyltin) sulfides, tri-n-butyltin laurate, tri-n-butyltin chloride, copper(I) oxide, triethyltin chloride, tri-n-butyl-(2-phenyl-4-chlorophenoxy)tin, tributyltin oxide, molybdenum disulfide, antimony oxide, polymeric butyl titanate, phenyl-(bispyridine)-bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylenebisthiocarbamate, zinc oxide, copper(I) ethylenebisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combination with the antifouling compositions according to the invention are:

algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

fungicides such as benzo[b]thiophenecarboxylic acid cyclohexylamide S,S-dioxide, dichlofluanid, fluorfolpet, 3-iodo-2-propynyl butylcarbamate, tolylfluanid and azoles such as azaconazole, cyproconazole, epoxyconazole, hexaconazole, metconazole, propiconazole and tebuconazole;

molluscicides such as fentin acetate, metaldehyde, methiocarb, niclosamid, thiodicarb and trimethacarb, Fe chelates;

or conventional antifouling active compounds such as 4,5-dichloro-2-octyl-4-isothiazolin-3-one, duiodomethylparatryl sulfone, 2-(N,N-dimethylthiocarbamoylthio)-5-nitrothiazyl, potassium, copper, sodium and zinc salts of 2-pyridinethiol 1-oxide, pyridine/triphenylborane, tetrabutyl-distannoxane, 2,3,5,6-tetrachloro4-(methylsulfonyl)-pyridine, 2,4,5,6-tetrachloroisophthalonitrile, tetramethylthiuram disulfide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as rosin to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga camaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

They are used in the household insecticides sector alone or in combination with other suitable active compounds such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

### PREPARATION EXAMPLES

### Example 1

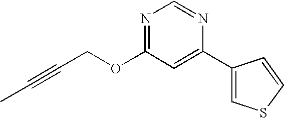

At room temperature (20° C.), 0.305 g (7.63 mmol) of sodium hydride (60% in oil) is initially charged in 35 ml of tetrahydrofuran, a solution of 0.36 g (5.09 mmol) of 2-butyn-1-ol in 15 ml of tetrahydrofuran is added dropwise and the mixture is stirred for 30 minutes. With ice-bath cooling, a solution of 1.0 g of 4-chloro-6-(3-thienyl)pyrimidine in 10 ml of tetrahydrofuran is then added dropwise, and the mixture is stirred for 12 hours. For work-up, the mixture is partitioned between saturated ammonium chloride solution and methyl t-butyl ether, and the organic phase is dried, concentrated under reduced pressure and purified further by column chromatography (silica gel RP-18, gradient acetonitrile/water).

This gives 0.5 g (40% of theory) of 4-(2-butyn-1-yl-oxy)-6-(3-thienyl)-pyrimidine log P 2.84

### Example 2

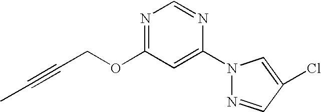

0.50 g (2.738 mmol) of 4-chloro-6-(2-butyn-1-yl-oxy)pyrimidine, 0.381 g (2.738 mmol) of 4-chloropyrazole hydrochloride and 1.963 g (6.024 mmol) of cesium carbonate in acetonitrile are heated under reflux for 12 hours. For work-up, the mixture is partitioned between aqueous citric acid solution and dichloromethane, and the organic phase is dried, concentrated under reduced pressure and purified further by column chromatography (initially silica gel, gradient cyclohexan/ethyl acetate, then silica gel RP-18, gradient acetonitrile/water).

This gives 0.369 g (54% of theory) of 4-butynyloxy-6-(4-chloropyrazol-1-yl)pyrimidine logP 3.65

The compounds of the formula (I) listed in Table 1 below, for example, can also be prepared analogously to Examples 1 and 2 and in accordance with the general description of the preparation process according to the invention.

TABLE 1

| | |
|---|---|
| | (I) |
| 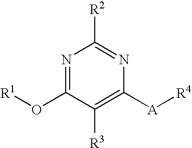 | |

Examples of compounds of the formula (I)

| Ex.- | | | | | | |
|---|---|---|---|---|---|---|
| No. | A | R¹ | R² | R³ | R⁴ | Physical data |
| 3 | — | 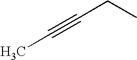 | H | H | 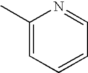 | logP = 2.31^{a)} |
| 4 | — | 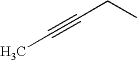 | H | H | 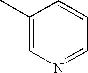 | |
| 5 | — | 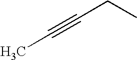 | H | H | 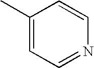 | |
| 6 | — | 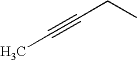 | H | H | 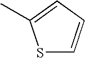 | logP = 2.95^{a)} |
| 7 | — | 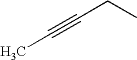 | H | H | 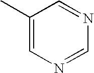 | |
| 8 | — | 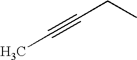 | H | H | 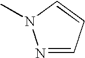 | logP = 2.55^{a)} |
| 9 | — | 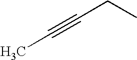 | H | H | 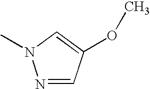 | logP = 2.78^{a)} |
| 10 | — | 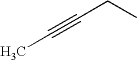 | H | H | 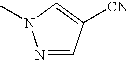 | logP = 2.68^{a)} |
| 11 | — | 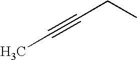 | H | H | 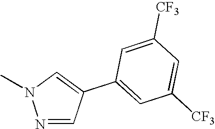 | logP = 5.33^{a)} |
| 12 | — | 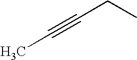 | H | H | 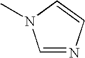 | logP = 1.00^{a)} |
| 13 | S | 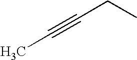 | H | H | 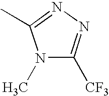 | logP = 2.35^{a)} |
| 14 | O | 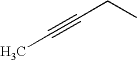 | H | H | 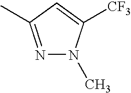 | logP = 3.19^{a)} |
| 15 | CH₂ | 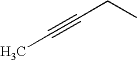 | H | H | 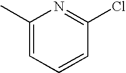 | |
| 16 | — | 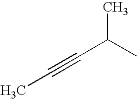 | H | F | 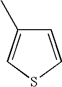 | |
| 17 | — | 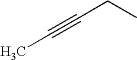 | H | NH₂ | 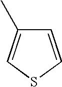 | |
| 18 | — | 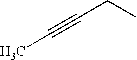 | H | CH₃ | 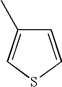 | |
| 19 | — | 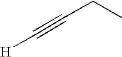 | H | H | 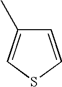 | logP = 2.47^{a)} |
| 20 | — | 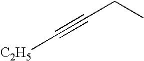 | H | H | 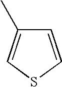 | logP = 3.32^{a)} |
| 21 | NH | 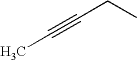 | H | H | 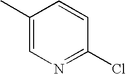 | |
| 22 | — | 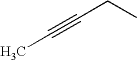 | H | H | 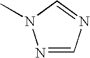 | |
| 23 | — | 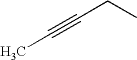 | H | F | 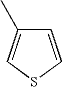 | |
| 24 | — | 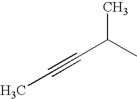 | H | H | 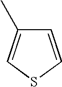 | logP = 3.18^{a)} |
| 25 | — | 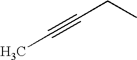 | H | H | 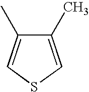 | logP = 3.26^{a)} |
| 26 | — | 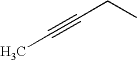 | H | H | 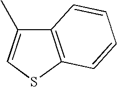 | logP = 3.92^{a)} |
| 27 | — | 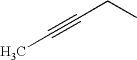 | H | CH₃ | 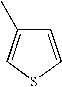 | |
| 28 | — | 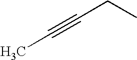 | H | CN | 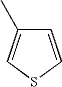 | |
| 29 | — | 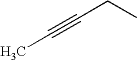 | H | NH₂ | 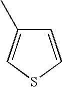 | |
| 30 | — | 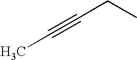 | CH₃ | H | 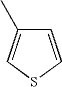 | logP = 1.81^{a)} |
| 31 | — | 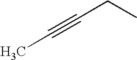 | NH₂ | H | 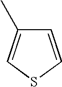 | logP = 2.94^{a)} |
| 32 | — | 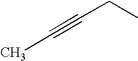 | tBu | H | 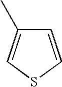 | logP = 5.74^{a)} |
| 33 | — | 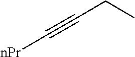 | H | H | 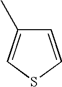 | logP = 3.79^{a)} |
| 34 | — | 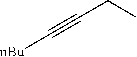 | H | H | 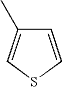 | logP = 4.29^{a)} |

The logP values given in the table were determined in accordance with EEC Directive 79/831 annex V.A8 by HPLC (High Performance Liquid Chromatography) on a reversed-phase column (C 18). Temperature: 43° C.
^{a)}Mobile phase for the determination in the acidic range (pH 2.3): 0.1% aqueous phosphoric acid, acetonitrile; linear gradient from 10% acetonitrile to 90% acetonitrile-corresponding measurement results are marked a) in Table 1.
^{b)}Mobile phase for the determination in the neutral range (pH 7.5): 0.01 molar aqueous phosphate buffer solution, acetonitrile; linear gradient from 10% acetonitrile to 90% acetonitrile-corresponding measurement results are marked b) in Table 1.

Calibration was carried out using unbranched alkan-2-ones (having 3 to 16 carbon atoms) with known logP values (determination of logP values by the retention times using linear interpolation between two successive alkanone).

The lambda max values were determined in the maxima of the chromatographic signals using the UV spectra from 200 nm to 400 nm.

Starting materials of the formula (II):

### Example (II-1)

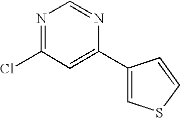

1.49 g (10 mmol) of 4,6-dichloropyrimidine are initially charged in 50 ml of 1,2-dimethoxyethane, 1.54 g (12 mmol) of 3-thiopheneboronic acid and 0.69 g (0.6 mmol) of tetrakis(triphenyl-phosphine)palladium(0) are added under argon, the mixture is stirred for 15 minutes, 50 ml of 20% strength sodium carbonate solution are added and the mixture is stirred at 80° C. for 4 hours. For work-up, the mixture is partitioned between citric acid solution and dichloromethane, the organic phase is dried and the solvent is carefully distilled off under reduced pressure.

This gives 2.1 g (94% of theory) of 4-chloro-6-(3-thienyl)pyrimidine

logP 2.31

Use Examples:

### Example A

Myzus Test (MYZUPE Spray Treatment)

| | | |
|---|---|---|
| Solvents: | 78 | parts by weight of acetone |
| | 1.5 | parts by weight of dimethylformamide |
| Emulsifier: | 0.5 | part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of Chinese cabbage (Brassica pekinensis) which are infested by all stages of green peach aphid (Myzus persicae) are sprayed with a preparation of active compound of the desired concentration.

After the desired period of time, the effect in % is determined. 100% means that all aphids have been killed; 0% means that none of the aphids have been killed.

In this test, for example, the following compounds of the Preparation Examples show good activity: see table

| | | MYZUPE | |
|---|---|---|---|
| | | spray treatment | |
| Example (I) | Structure | g/ha | % 5 d |
| 6 | 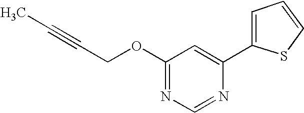 | 500 | 90 |
| 8 | 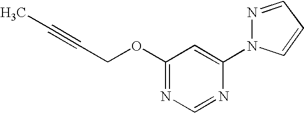 | 500 | 90 |
| 3 | 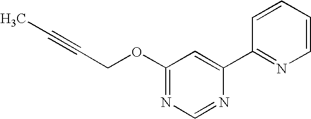 | 500 | 80 |
| 25 | 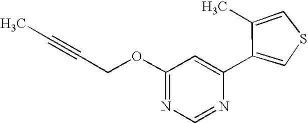 | 500 | 80 |

### Example B

Aphis Gossypii Test (APHIGO)

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cotton leaves (Gossypium hirsutum) which are heavily infested by the cotton aphid (Aphis gossypii) are treated by being dipped into the preparation of active compound of the desired concentration.

After the desired period of time, the kill in % is determined. 100% means that all aphids have been killed; 0% means that none of the aphids have been killed.

In this test, for example, the following compound of the Preparation Example shows good activity: see table

| | | APHIGO | |
|---|---|---|---|
| Example (I) | Structure | ppm | % 6 d |
| 25 | 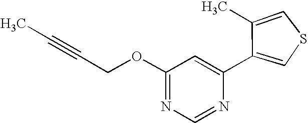 | 100 | 80 |

### Example C

Myzus Test; Oral (MYZUPE O)

| | |
|---|---|
| Solvent: | 80 parts by weight of acetone |

To produce a suitable preparation of active compound, 1 part by weight of active compound is mixed with the stated amounts of solvent, and the concentrate is diluted with water to the desired concentration.

Containers are populated with all stages of the green peach aphid (Myzus persicae), treatment is by sucking at the preparation of active compound of the desired concentration.

After the desired period of time, the effect in % is determined. 100% means that all aphids have been killed; 0% means that none of the aphids have been killed.

In this test, for example, the following compound of the Preparation Examples shows good activity: see table

| | | MYZUPE | |
|---|---|---|---|
| | | O | |
| Example (I) | Structure | ppm | % 6 d |
| 19 | 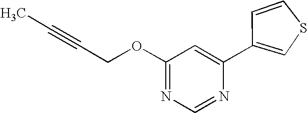 | 1000 | 100 |

### Example D

Myzus Persicae (MYZUPE U)

| | |
|---|---|
| Solvents: | 1% of N-methylpyrolidone (NMP) |
| | 1% of diacetone alcohol |
| Dye: | Brillant sulfoflavin for staining the water |

To produce a suitable preparation of active compound, the active compound is mixed with the stated amounts of solvent, and the concentrate is diluted with stained water to the desired concentration.

The Myzus persicae are provided with a preparation of active compound of the desired concentration for ingestion.

After the desired period of time, the effect in % is determined. 100% means that all aphids have been killed; 0% means that none of the aphids have been killed.

In this test, for example, the following compounds of the Preparation Examples show good activity:

| | | MYZUPE | |
|---|---|---|---|
| | | U | |
| | | | % 6-7 |
| Example (I) | Structure | ppm | d |
| 19 | 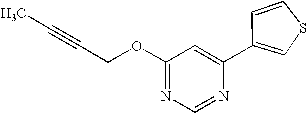 | | |
| 12 | 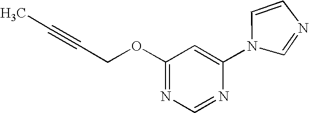 | 30 | 100 |

1. A compound of the formula (I), 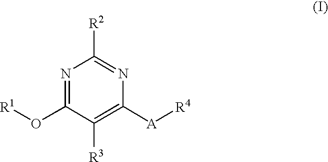
in which
A represents a single bond, O (oxygen), S (sulfur), NH, N(C₁-C₄-alkyl), C═O, C═N—O—R, where R represents hydrogen or C₁-C₄-alkyl, or represents optionally hydroxyl- or halogen-substituted straight-chain or branched alkanediyl having 1 to 4 carbon atoms,
R¹ represents optionally halogen-substituted straight-chain or branched alkynyl having 2 to 10 carbon atoms,
R² represents hydrogen, amino, halogen or halogen-substituted straight-chain or branched alkyl having 1 to 4 carbon atoms,
R³ represents hydrogen, amino, halogen or optionally halogen-substituted straight-chain or branched alkyl having 1 to 4 carbon atoms, and
R⁴ represents a monocyclic or bicycyclic heteroaromatic group having up to 9 carbon atoms and at least one heteroatom selected from the group consisting of N, O, and S, wherein said monocyclic or bicycyclic heteroaromatic group is optionally substituted by nitro, hydroxyl, mercapto, amino, cyano, carboxyl, carbamoyl, thiocarbamoyl, halogen, or by in each case optionally hydroxyl-, cyano- or halogen-substituted C₁-C₆-alkyl, C₁-C₆-alkyl-carbonyl, C₁-C₆-alkoxy, C₁-C₆-alkoxy-carbonyl, C₁-C₆-alkylthio, C₁-C₆-alkylsulfinyl, C₁-C₆-alkylsulfonyl, C₁-C₆-alkylamino, C₁-C₆-alkyl-aminocarbonyl, di-(C₁-C₆-alkyl)-amino, di-(C₁-C₆-alkyl)-amino-carbonyl or di-(C₁-C₆-alkyl)-amino-sulfonyl.

2. A compound of the formula (I) according to claim 1, wherein
A represents a single bond, O (oxygen), S (sulfur), NH, N(methyl), N(ethyl), N(propyl), C═O, C═N—O—R, where R represents hydrogen, methyl or ethyl, or represents optionally hydroxyl-, fluorine- or chlorine-substituted straight-chain or branched alkanediyl having 1 to 3 carbon atoms,
R¹ represents optionally fluorine-, chlorine-, bromine- or iodine-substituted straight-chain or branched alkynyl having 3 to 6 carbon atoms,
R² represents hydrogen, amino, fluorine, chlorine, bromine, iodine or fluorine- or chlorine-substituted straight-chain or branched alkyl having 1 to 3 carbon atoms,
R³ represents hydrogen, amino, fluorine, chlorine, bromine, iodine or optionally fluorine- or chlorine-substituted straight-chain or branched alkyl having 1 to 3 carbon atoms,
R⁴ represents a monocyclic or bicycyclic heteroaromatic group having up to 9 carbon atoms and at least one heteroatom selected from the group consisting of N, O and S wherein said monocyclic or bicycyclic heteroaromatic group is optionally substituted by nitro, hydroxyl, mercapto, amino, cyano, carboxyl, carbamoyl, thiocarbamoyl, fluorine, chlorine, bromine, iodine; or by in each case optionally hydroxyl-, cyano-, fluorine-, chlorine- or bromine-substituted C₁-C₅-alkyl, C₁-C₅-alkyl-carbonyl, C₁-C₅-alkoxy, C₁-C₅-alkoxy-carbonyl, C₁-Cs-alkylthio, C₁-C₅-alkylsulfinyl, C₁-C₅-alkylsulfonyl, C₁-C₅-alkylamino, C₁-C₅-alkyl-aminocarbonyl, di-(C₁-C₅-alkyl)-amino, di-(C₁-C₅-alkyl)-amino-carbonyl or di-(C₁-C₅-alkyl)-amino-sulfonyl.

3. A compound of formula (I) according to claim 1, wherein
A represents a single bond, O (oxygen), S (sulfur), NH, N(methyl), methylene, ethane-1,1-diyl (ethylidene) or ethane-1,2-diyl (dimethylene),
R¹ represents optionally fluorine-, chlorine-, bromine- or iodine-substituted 2-propyn-1-yl, 2-butyn-1-yl, 3-butyn-2-yl or 2-pentyn-1-yl,
R² represents hydrogen, fluorine, chlorine, bromine or fluorine- or chlorine-substituted methyl,
R³ represents hydrogen, fluorine, chlorine, bromine or optionally fluorine- or chlorine-substituted methyl,
R⁴ represents a monocyclic heteroaromatic group selected from the group consisting of furyl, thienyl, pyrrolyl, pyrazolyl, imidazolyl, oxazolyl, isoxazolyl, thiazolyl, isothiazolyl, oxadiazolyl, thiadiazolyl, triazolyl, pyridinyl, pyrimidinyl, pyrazinyl, pyridazinyl and triazinyl, wherein said monocyclic heteroaromatic group is optionally substituted by nitro, hydroxyl, mercapto, amino, cyano, carboxyl, carbamoyl, thiocarbamoyl, fluorine, chlorine, bromine, iodine; or by in each case optionally hydroxyl-, cyano-, fluorine- or chlorine-substituted methyl, ethyl, n- or i-propyl, n-, i-, s- or t-butyl, acetyl, propionyl, n- or i-butyroyl, methoxy, ethoxy, n- or i-propoxy, methoxycarbonyl, ethoxycarbonyl, n- or i-propoxycarbonyl, n-, i-, s- or t-butoxycarbonyl, methylthio, ethylthio, n- or i-propylthio, methylsulfinyl, ethylsulfinyl, methylsulfonyl, ethylsulfonyl, methylamino, ethylamino, n- or i-propylamino, n-, i-, s- or t-butylamino, methylaminocarbonyl, ethylaminocarbonyl, n- or i-propylaminocarbonyl, dimethylamino, diethylamino, dimethylaminocarbonyl, diethylaminocarbonyl, dimethylaminosulfonyl or diethylaminosulfonyl.

4. A compound of formula (I) according to claim 1, wherein
A represents a single bond, O (oxygen), S (sulfur), NH, N(methyl) or methylene,
R¹ represents 2-propyn-1-yl, 2-butyn-1-yl or 2-pentyn-1-yl,
R² represents hydrogen,
R³ represents hydrogen, fluorine, chlorine or methyl,
R⁴ represents a monocyclic heteroaromatic group selected from the group consisting of furyl, thienyl, pyrrolyl, pyrazolyl, imidazolyl, oxazolyl, isoxazolyl, thiazolyl, isothiazolyl, oxadiazolyl, thiadiazolyl, triazolyl, pyridinyl, pyrimidinyl, pyrazinyl, pyridazinyl and triazinyl, wherein said monocyclic heteroaromatic group is optionally substituted by nitro, cyano, carboxyl, carbamoyl, thiocarbamoyl, fluorine, chlorine, bromine; or by in each case optionally cyano-, fluorine- or chlorine-substituted methyl, ethyl, n- or i-propyl, n-, i-, s- or t-butyl, acetyl, propionyl, n- or i-butyroyl, methoxy, ethoxy, n- or i-propoxy, methoxycarbonyl, ethoxycarbonyl, n- or i-propoxycarbonyl, methylthio, ethylthio, n- or i-propylthio, methylsulfinyl, ethylsulfinyl, methylsulfonyl, ethylsulfonyl, methylamino, ethylamino, n- or i-propylamino, methylaminocarbonyl, ethylaminocarbonyl, n- or i-propylaminocarbonyl, dimethylamino, diethylamino, dimethylaminocarbonyl or dimethylaminosulfonyl.

5. A process for preparing a compound of formula (I) according to claim 1, comprising
a) reacting a pyrimidine of the formula (II) 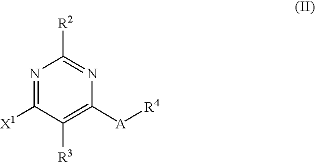
in which
X¹ represents halogen or C₁-C₄-alkylsulfonyl,
with an alkynol of the formula (III) 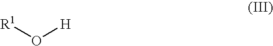
or an alkali metal salt thereof, optionally in the presence of one or more reaction auxiliaries and optionally in the presence of one or more diluents, or
b) reacting a pyrimidine of the formula (IV) 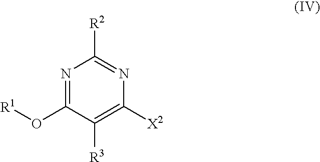
in which
X² represents halogen or C₁-C₄-alkylsulfonyl,
with a nucleophilic compound of the formula (V) 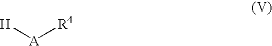
or an alkali metal salt thereof,
optionally in the presence of one or more reaction auxiliaries and optionally in the presence of one or more diluents.

6. A composition, comprising at least one compound of the formula (I) according to claim 1 and an extender or a surfactant or combinations thereof.

7. A method for controlling pests, comprising contacting said pests or their habitat with a compound of formula (I) according to claim 1.

8. (canceled)

9. A method for controlling pests, comprising contacting said pests or their habitat with a composition according to claim 6.
